(19) United States
(12) Patent Application Publication
(10) Pub. No.: US 2025/0270652 A1
(43) Pub. Date: Aug. 28, 2025

(54) Method for Evaluating Clinical Relevance of Genetic Variance

(71) Applicant: Heligenics Inc., Las Vegas, NV (US)

(72) Inventors: Martin Roy SCHILLER, Henderson, NV (US); Elizabeth Joy VALENTE, Henderson, NV (US); Lancer BROWN, Henderson, NV (US); Christopher John GIACOLETTO, Las Vegas, NV (US)

(21) Appl. No.: 19/049,231

(22) Filed: Feb. 10, 2025

Related U.S. Application Data
(63) Continuation of application No. PCT/US2023/071962, filed on Aug. 9, 2023.
(60) Provisional application No. 63/370,837, filed on Aug. 9, 2022.
(60) Provisional application No. 63/375,369, filed on Sep. 12, 2022.

(51) Int. Cl. C12Q 1/6886; C12N 15/10; C12N 15/86; C12Q 1/6869; C40B 40/10; G01N 33/574

(52) U.S. Cl. CPC C12Q 1/6886; C12N 15/1037; C12N 15/86; C12Q 1/6869; C40B 40/10; G01N 33/574; C12N 2740/15043; C12N 2750/14143; C12Q 2600/156; G01N 2333/82

(57) ABSTRACT

Disclosed herein are systems and methods for screening variant libraries for activity. Also disclosed herein are high throughput methods for identifying candidate variant having gain of function biological activity in an assay.

### CROSS REFERENCE

This application is a continuation of PCT Application No. PCT/US2023/071962, filed Aug. 9, 2023, which claims the benefit of U.S. Provisional Application No. 63/370,837, filed Aug. 9, 2022, and U.S. Provisional Application No. 63/375,369, filed Sep. 12, 2022, the entire contents of which are incorporated herein by reference.

### SEQUENCE LISTING

The instant application contains a Sequence Listing which has been submitted electronically in ST.26 (xml) format and is hereby incorporated by reference in its entirety. Said ST.26 (xml) copy, created on Sep. 14, 2023, is named 218963-702601_SL.xml and is 5,176 bytes in size.

### SUMMARY

Disclosed herein is a method of identifying a Erb-B2 Receptor Tyrosine Kinase 2 (ERBB2) polypeptide variant implicated in a cancer, the method comprising: (a) providing a plasmid library for expression of a library of ERBB2 polypeptide variants, wherein each plasmid in the plasmid library comprises: (i) a promoter; (ii) a polynucleotide sequence encoding an ERBB2 polypeptide variant, from the library of ERBB2 polypeptide variants, that is operably coupled to the promoter, wherein each ERBB2 polypeptide variant in the library of ERBB2 polypeptide variants independently and substantially comprises a single amino acid substitution in a region of the ERBB2 polypeptide ranging from residue 679 to residue 992 of SEQ ID NO: 1; wherein the library of ERBB2 polypeptide variants comprises ERBB2 polypeptide variants that collectively have an amino acid substitution of substantially all 20 amino acids at substantially every amino acid residue in the region of the ERBB2 polypeptide ranging from residue 679 to residue 992 of SEQ ID NO: 1; and (iii) a barcode, wherein each plasmid in the plasmid library independently has a different barcode associated with the polynucleotide sequence, in the plasmid, encoding an ERBB2 polypeptide variant; (b) contacting a plurality of mammalian cells with the plasmid library, wherein the contacting results in expression of a single ERBB2 polypeptide variant among the library of ERBB2 polypeptides on a surface of a single mammalian cell among the plurality of mammalian cells, thereby making a plurality of mammalian cells expressing the library of ERBB2 polypeptide variants, wherein a subset of the plurality of mammalian cells expressing the library of ERBB2 polypeptide variants has an ERBB2 polypeptide variant that is phosphorylated to a greater extent than a wildtype ERBB2 polypeptide expressed on the surface of a mammalian cell, and wherein the ERBB2 polypeptide variant that is phosphorylated to a greater extent than the wildtype ERBB2 polypeptide is the ERBB2 variant that is implicated in the cancer; (c) identifying the subset of mammalian cells expressing the ERBB2 polypeptide variant that is phosphorylated to a greater extent than the wildtype ERBB2 polypeptide; and (d) sequencing the barcode of the plasmid present in each mammalian cell of the subset of mammalian cells, thereby identifying the ERBB2 polypeptide variant implicated in the cancer. In some embodiments, the cancer comprises a carcinoma. In some embodiments, the cancer comprises an ovarian cancer, a stomach cancer, a bladder cancer, a salivary cancer, or a lung cancer. In some embodiments, the method further comprises detecting the presence of the ERBB2 polypeptide variant identified in (d) from a sample obtained from a subject. In some embodiments, the method further comprises diagnosing the subject as having the cancer, or being at risk of developing the cancer. In some embodiments, the method further comprises administering an anticancer treatment to the subject, optionally based on the presence of the ERBB2 polypeptide variant identified in (d) from the sample obtained from the subject. In some embodiments, the anticancer treatment is effective against a cancer cell expressing the ERBB2 polypeptide variant. In some embodiments, the identifying in (c) comprises contacting the plurality of mammalian cells expressing the library of ERBB2 polypeptide variants with an agent that binds to phosphorylated ERBB2. In some embodiments, the agent that binds to phosphorylated ERBB2 is an antibody directed against phosphorylated ERBB2. In some embodiments, the identifying of (c) further comprises performing an immunoassay using the antibody directed against phosphorylated ERBB2. In some embodiments, the identifying of (c) further comprises performing cell sorting based on the immunoassay. In some embodiments, the cell sorting is fluorescence activated cell sorting. In some embodiments, the promoter is an inducible promoter. In some embodiments, the inducible promoter is a doxycycline-inducible promoter. In some embodiments, a plasmid in the library of plasmids is a viral plasmid. In some embodiments, the viral plasmid is a lentiviral plasmid or an adeno-associated viral plasmid. In some embodiments, the method further comprises packaging the viral plasmid in a viral capsid prior to the contacting of (b), thereby producing virion that contain the viral plasmid. In some embodiments, the contacting of (b) comprises contacting a mammalian cell of the plurality of mammalian cells with the virion. In some embodiments, the plurality of mammalian cells comprise HEK 293 cells. In some embodiments, the sequencing comprises next generation sequencing. In some embodiments, the method further comprises stably-expressing the ERBB2 polypeptide variant implicated in cancer identified in (d) in a mammalian cell and measuring the activity of the ERBB2 polypeptide when stably expressed. In some embodiments, the method further comprises comparing the activity of the ERBB2 polypeptide when stably expressed to stably expressed wildtype ERBB2. In some embodiments, at least 80% of the variants identified in (d) display higher activity when stably expressed, as compared to stably expressed wildtype ERBB2. In some embodiments, the method further comprises entering the ERBB2 polypeptide variant implicated in cancer identified in (d) into a database.

Also disclosed herein is a database that comprises an ERBB2 polypeptide variant identified by the method described herein.

Also disclosed herein is a composition that comprises an ERBB2 polypeptide variant identified by the method described herein.

### BRIEF DESCRIPTION OF THE DRAWINGS

Novel features of exemplary embodiments are set forth with particularity in the appended claims. A better understanding of the features and advantages will be obtained by reference to the following detailed description that sets forth illustrative embodiments, in which the principles of the disclosed systems and methods are utilized, and the accompanying drawings of which:

FIG. 1A is a schematic illustrating exemplary steps in the method described herein for pERBB2 activation. Propagation of the recombined cells under poison selection. Cell sorting based on GFP reporter expression or immunostaining. gDNA is isolated, and a targeted ERBB2 amplicon library is prepared and sequenced by NGS.

FIG. 1B is a schematic illustrating an ERBB2 activation assay used in the method described herein.

FIG. 2 is a Table illustrating various project design parameters.

FIG. 3 is a Table illustrating a summary of the project statistics.

FIG. 4 is a Table illustrating variants analyzed with the method described herein including Activity scores (GML Score) and confidence values (P Value). WT=wild-type; GOF=gain of function; LOF=loss of function.

FIG. 5 is a Table illustrating assay validation statistics of the method described herein.

FIG. 6 is a heatmap illustrating sequence reads for each variated amino acid for each position in Erbb2. The color gradient represents the number of associated reads. White boxes are the wild-type amino acids and grey boxes are null values. Darker shades are associated with greater read counts.

FIG. 7 is a heatmap illustrating the number of barcodes for each amino acid variant of each position in ERBB2. The color gradient represents the number of associated barcodes. White boxes are the wild-type amino acids and grey boxes are null values. Darker shades are associated with greater read counts.

FIG. 8 is a pie chart illustrating a summary of the reads for ERBB2 variants. The variant distribution across various levels of read depths were quantified.

FIG. 9 is a pie chart illustrating a summary of the number of barcode reads for ERBB2 variants. The variant distribution across various levels of barcode depths were quantified.

FIG. 10 is a heatmap illustrating the number of barcodes for each amino acid variant of each position in ERBB2. The color gradient represents the relative activity of the variant. Red variants are RF variants and green variants are GOF variants. White boxes have wild-type level activity. Black boxes are the reference amino acids and grey boxes are null values.

FIG. 11 is a heatmap illustrating the number of barcodes for each amino acid variant of each position in ERBB2. Statistically relevant activities are plotted (p<0.05). Red colors are RF variants and green colors are GOF variants. White boxes have wild type level activity, black boxes are the Ref amino acids and grey boxes are null values.

FIG. 12 is an overlayed heatmap for both p-value and activity for variants illustrating variant amino acids for each position in ERBB2. The color gradient represents the relative activity of the variant. White boxes have wild type level activity, black boxes are the Ref amino acids and grey boxes are null values. Variant activity level are colored on the gradient key. Red colors are RF variants and green colors are GOF variants. The number of small blue boxes indicates the p value (p<0.05 (no boxes); <0.05 (1 box); <0.001 (2 box); <0.0001 (3 box); <0.00001 (4 box).

FIG. 13 is a pie chart illustrating variant classifications. The number of statistically relevant variants (p<0.05) were quantified for each category and are shown.

FIG. 14 are flow sort profiles illustrating individual clones in cell colonies and that were tested through the pERBB2 assay prior to performing the GML experiment. Relative fluorescence is shown above for each control.

FIG. 15 are 3D structural renderings illustrating HER2 variant impact on function. All surface maps are on the wild-type ERBB2 structure (PDB:3PPO) with one mem er of each pair rotated 180 about the Y axis: A) Amino acid positions on erbb2 backbone; B) Activation loop of ERBB2: C) ATP binding site; D) hyperactivating variants; E) hyperactivating variants from the method described herein; F) phosphorylation site of ERBB2; G) LOF variants; H) RF variants from the method described herein; and I) ubiquitination sites of ERBB2. For all plots, at least one substitution at the positions shown had an associated activity.

FIGS. 16A and 16B are flow sort profiles illustrating individual clones in cell colonies and that were tested through the pERBB2 assay prior to performing the GML experiment. Relative fluorescence is shown above for each control.

### DETAILED DESCRIPTION

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

Conditional language, such as “can,” “could,” “might,” or “may,” unless specifically stated otherwise, or otherwise understood within the context as used, is generally intended to convey that certain implementations include or do not include, certain features, elements, and/or steps. Thus, such conditional language is not generally intended to imply that features, elements, and/or steps are in any way required for one or more implementations.

Conjunctive language, such as the phrase “at least one of X, Y, and Z.” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

As used herein, the term, “about” or “approximately,” means within an acceptable error range for the particular value and includes a range of up to 10% of a given value. Where particular values are described in the application and claims, unless otherwise stated the term “about” meaning within an acceptable error range for the particular value should be assumed.

The term “substantially” refers to a qualitative condition that exhibits at least 70% of a total range or degree of a feature or characteristic of interest.

As used herein, the terms “ERBB2” and “Her2” are used interchangeably to refer to the same polypeptide or same gene encoding the same polypeptide.

The term “operably coupled” refers to functional linkage between a regulatory sequence and a nucleic acid sequence resulting in expression of the latter

Although this disclosure has been described in terms of certain implementations and uses, other implementations and other uses, including implementations and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various implementations. All possible combinations and subcombinations of elements and components described herein are intended to be included in this disclosure. No single feature or group of features is necessary or indispensable.

Any portion of any of the steps, processes, structures, and/or devices disclosed in one implementation or example in this disclosure can be combined or used with (or instead of) any other portion of any of the steps, processes, structures, and/or devices disclosed or illustrated in a different implementation, flowchart, or example. The implementations and examples described herein are not intended to be discrete and separate from each other. Combinations, variations, and some implementations of the disclosed features are within the scope of this disclosure.

While operations may be described in the specification in a particular order, such operations need not be performed in the particular order described or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

### Overview

Disclosed herein is a method for identifying ERBB2 variants that are implicated in the progression of a cancer associated with mutations in ERBB2. ERBB2 is a receptor tyrosine kinase with intrinsic tyrosine kinase activity. Currently, there is no known substrate for the receptor, and as such it is believed that the ERBB2 extracellular domain remains in the “open position” similar to other members of the mammalian EGFR family when unbound to their natural ligand. Being in the “open” state, ERBB2 is capable of binding to other mammalian EGFR family members readily.

EGFR amplification and/or overexpression is believed to be implicated in a number of cancers, such as ovarian cancer, stomach cancer, bladder cancer, salivary cancer, and lung cancer. Further, elevated phosphorylation of ERBB2 via mutations that increase intrinsic tyrosine kinase activity has been implicated in the progression of such cancers, and potentially results in drug resistance of certain cancer cells expressing the mutated ERBB2 that result in elevated phosphorylation. In some embodiments, the mutation is with respect to the ERBB2 polypeptide of SEQ ID NO: 1.

SEQ ID NO: 1
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

In some cases, these mutations are mutations in the tyrosine kinase domain (residues 715-992 of SEQ ID NO: 1), the junction membrane (JM) region (residues 679-714 of SEQ ID NO: 1), or both. However, a systematic method to determine which residues in ERBB2, when mutated, results in the increase in autophosphorylation implicated in the progression of cancer. Accordingly, the method of the current disclosure provides this systematic approach by saturation mutagenizing the amino acid at residues 679-992 of SEQ ID NO: 1, thus producing a library of ERBB2 variants substantially covering the entire sequence space. The library is then screened directly in a mammalian cell in order to recapitulate the native environment of the ERBB2 variant. Finally, the degree of phosphorylation for each ERBB2 variant present on the surface of the mammalian cell is directly measured, thus allowing for binning of ERBB2 variants based on their degree of phosphorylation. By comparing the degree of phosphorylation to that of wildtype or benchmark controls, a comprehensive profile of ERBB2 variants implicated in cancer was constructed. This approach provides identification of novel ERBB2 variants that are implicated in the progression of cancer. Using the profile of ERBB2 variants implicated in cancer generated herein as a guide, novel methods of detecting and/or treating cancer are also provided in which one or more ERBB2 variants identified using the method described herein can be detected in a sample obtained from a subject, thereby diagnosing the subject as having the cancer or being at risk of developing the cancer.

### Method of Screening

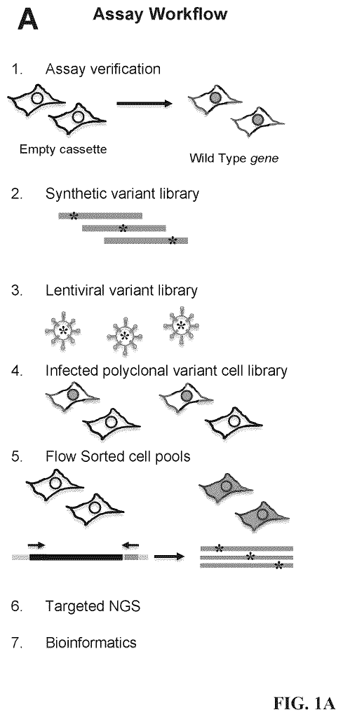

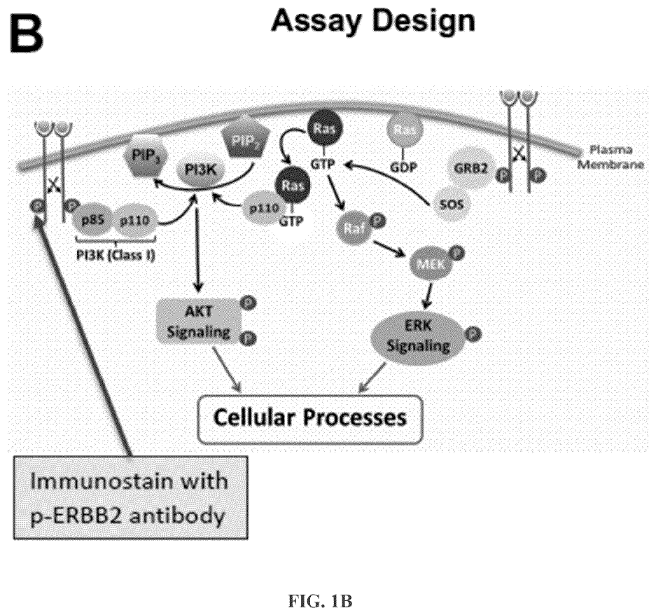

Disclosed herein is a method of identifying Erb-B2 Receptor Tyrosine Kinase 2 (ERBB2) polypeptide variants implicated in cancer. FIG. 1A provides a general overview of an embodiment of the method described herein. In some embodiments, a method described herein can include one or more of steps 1-7 recited in FIG. 1A. In some embodiments, the method can comprise one or more of:
(i) Assay verification;
(ii) Production of a synthetic variant library;
(iii) Generation of a lentiviral variant library;
(iv) Infecting cells with the lentiviral variant library;
(v) Cell sorting based on the assay readout;
(vi) Identification of variants using targeted next generation sequencing; and
(vii) Biochemical/bioinformatic characterization of identified variants.

In some embodiments, the method comprises one or more of:
(a) providing a plasmid library for expression of a library of ERBB2 polypeptide variants, wherein each plasmid in the plasmid library comprises one or more of: (i) a promoter; (ii) a polynucleotide sequence encoding an ERBB2 polypeptide variant from the library of ERBB2 polypeptide variants that is operably coupled to the promoter, wherein each ERBB2 polypeptide variant in the library of ERBB2 polypeptide variants independently and substantially comprises a single amino acid substitution in a region of the ERBB2 polypeptide ranging from residue 679 to residue 992 of SEQ ID NO: 1; wherein the library of ERBB2 polypeptide variants comprises ERBB2 polypeptide variants that collectively have an amino acid substitution of substantially all 20 amino acids at substantially every amino acid residue in the region of the ERBB2 polypeptide ranging from residue 679 to residue 992 of SEQ ID NO: 1; and (iii) a barcode, wherein each plasmid in the plasmid library independently have different barcodes associated with the polynucleotide sequence encoding the ERBB2 polypeptide variants;
(b) contacting a plurality of mammalian cells with the plasmid library, wherein the contacting results in expression of a single ERBB2 polypeptide variant among the library of ERBB2 polypeptides on a surface of a single mammalian cell among the plurality of mammalian cells, thereby making a plurality of mammalian cells expressing the library of ERBB2 polypeptide variants, wherein a subset of the plurality of mammalian cells expressing the library of ERBB2 polypeptide variants have ERBB2 polypeptide variants that are phosphorylated to a greater extent than a wildtype ERBB2 polypeptide expressed on the surface of a mammalian cell, and wherein the ERBB2 polypeptide variants that are phosphorylated to a greater extent than the wildtype ERBB2 polypeptide are ERBB2 variants that are implicated in cancer;
(c) contacting the plurality of mammalian cells expressing the library of ERBB2 polypeptide variants with an agent that binds to phosphorylated ERBB2;
(d) identifying the subset of mammalian cells expressing the ERBB2 polypeptide variants that are phosphorylated to a greater extent than the wildtype ERBB2 polypeptide based on binding of the agent to the phosphorylated ERBB2 expressed on the surface of the subset of mammalian cells; and
(e) sequencing the barcode of the plasmid present in each mammalian cell of the subset of mammalian cells, thereby identifying the ERBB2 polypeptide variants implicated in cancer.

In some embodiments, each plasmid has the same promoter. In some embodiments, each plasmid has a different promoter. In some embodiments, the promoter is a constitutive promoter such as SV40, CMV, UBC, EF1A, PGK or CAGG. In some embodiments, the promoter is an inducible promoter such as doxycycline or tetracycline. Embodiments described herein utilize an assay to detect variants of ERBB2 implicated in cancer. FIG. 1B provides an illustration of ERBB2 signaling pathways. Without wishing to be bound by theory, auto-phosphorylated ERBB2 may be implicated in the AKT and ERK signaling pathways, with aberrant, abnormally high phosphorylation of ERBB2 resulting in the progression of cancers described herein. As such, the presence of phosphorylated ERBB2 using an agent that binds to phosphorylated ERBB2 can be used in an assay to screen for ERBB2 variants with abnormally high phosphorylation, which would thus be implicated in the progression of cancer.

Using the methods described herein, comprehensive profiles can be generated to model gene variants (e.g., phospho-her2), based on, for example, the assay in mammalian cell culture depicted in FIG. 1B (e.g., a method for her2 tyrosine kinase domain activation of phospho-her2). A plasmid library can be constructed encoding these variants, which is then analyzed using the methods described herein to produce a comprehensive variant effect on gene activity, as well as a variant activity profile.

The high-throughput cellular molecular function assay methods described herein have several advantages over existing methods. For example, performing saturation mutagenesis of entire regions of the ERBB2 polypeptide allows for all variations or nearly all variations to be directly measured in the biologically-native environment, rather than inferring activity from differences between pre-screen and screened samples. Further, the methods described herein can utilize plasmids that each encode an individual ERBB2 polypeptide variant and have individual barcodes that are associated with the particular ERBB2 polypeptide variant. Because of the barcoding of individual molecules and high throughput performance at the single cell assay level, using methods described herein allows for signal averaging large numbers of individual measurements yielding robust reproducibility, high accuracy, and a statistic for reliability of the activity for each variant. Furthermore, all ERBB2 variants are assayed under standardized conditions in the same cells with the same genetic background, which produces a consistent data set.

The present disclosure exemplifies this method as it relates to production of ERBB2 (the protein produced from the Erbb2 gene), including the constitutively active YVMA indel variant that induces ERBB2 phosphorylation for activation of the MAPK pathway. Detecting phosphorylated ERBB2 is direct measure of ERBB2/Her2 activity. Alternatively, indirect measurements of ERBB2/Her2 activity can be performed by measuring p-ERK activation. However, p-ERK activation can also arise from other pathways and mitogen receptors, which requires more robust controls and has a lower signal to noise ratio.

Utilizing embodiments provided herein that directly detect ERBB2/Her2 activity through production of phosphorylated ERBB2/Her2 can be coupled to downstream/global measures of oncogenicity by cell proliferation, and can utilize known variants as controls to increase the pathway-specific accuracy of the assays, which can, at least in part, help reduce ambiguity in variant interpretation. In some instances, quantitation of the oncogenic potential of known or identified variants using a low-throughput phospho-Her2 flow cytometry assay (in triplicate) can be coupled with the method described herein to provide standardization of the methods described herein.

The methods provided herein are modular high-throughput one-pot assay systems for measuring molecular functions of a large number of genetic variants at once with high accuracy, such as hundreds or thousands of variants. In some instances, the methods provided herein measure millions of genetic variants. In some applications, the high-throughput methods provided herein have an overall accuracy of more than 75% as compared to activity measurements using, for example, low-throughput flow cytometry. In some embodiments, the accuracy of the methods described herein is about 80%, about 82%, about 84%, about 86%, about 88%, or about 90%, as compared to activity measurements using, for example, low-throughput flow cytometry. In some embodiments, the accuracy of the methods described herein for identifying variants having gain-of-function activity (i.e., higher activity as compared to wild-type) is about 80%, about 82%, about 84%, about 86%, about 88%, or about 90%, as compared to activity measurements using, for example, low-throughput flow cytometry. In some embodiments, accuracy of the methods described herein have perfect or near perfect concordance with other standardized assays (100% accuracy or near-100% accuracy).

### Barcoded Variant Library

A plasmid can be made by introducing compatible restriction enzyme sites (e.g., EcoRI, SalI, and AsiSI) and inserting a desired clone of the ERBB2/Her2 variant library. An ERBB2/Her2 variant encoding can be PCR amplified from a template with a Polymerase and cloned into the digested plasmid. In some embodiments, the plasmid is a retrovirus. In some embodiments, the plasmid is a lentiviral plasmid. In some embodiments, the plasmid is an AAV plasmid. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, avian AAV, bovine AAV, canine AAV, equine AAV, or ovine AAV.

In some embodiments, the plasmid comprises DNA. In some embodiments, the plasmid comprises RNA. In some examples, the plasmid comprises circular double-stranded DNA. In some examples, the plasmid may be linear. Various selectable markers can be used to select for plasmid transduction such as puromycin or blasticidin S resistance genes. For example, the selectable marker and Her2 variant amplicons can be fused by inverse PCR using a polymerase. The fused amplicons are then cloned into the plasmid digested with a compatible restriction enzyme.

In some embodiments, a double stranded (ds) DNA library containing Her2 cDNAs with sequences for all the possible single amino acid variants are synthesized. The dsDNA from each well can be pooled and a single round of overlap PCR extension appended random-mers oligonucleotides to the 3′ untranslated region. The synthesized dsDNA library has a 3′-overhang sequence after the stop codon that overlaps with the 5′ overhang sequence upstream of the random-mers oligonucleotide sequence. The pooled ds DNA library and the random oligomer can be mixed in 1:2, 1:5, 1:10, 1:20, and 1:50 molar ratio, denatured, and annealed. Hybridized DNA can be extended with a DNA Polymerase for one cycle of PCR. The PCR reaction mix can then be treated with an exonuclease and purified.

The purified DNA is digested with compatible restriction enzymes and ligated into the digested plasmid with a ligase. Ligation reactions can be pooled, purified, and dialyzed. The purified ligation reaction mixture can be electroporated into electrocompetent cells, plated, and incubated. Transformants can be scrapped from the plates and the plasmid library from the pooled cell suspension can be isolated.

Viral libraries (e.g., lentiviral libraries) are produced in transformant compatible cells (e.g., LentiX 293T cells). In some embodiments, tens of thousands of cells to billions of cells are seeded into petri dishes. In some embodiments, approximately 3 million LentiX293T cells are seeded in a petri dish and grown in complete media (e.g., DMEM+10% Fetal Calf serum). In some embodiments, the plasmid includes one or more regulatory elements. In some embodiments, the plasmid comprises packaging elements used in transformation of the viral plasmid. For example, the plasmid library; a vector encoding packaging elements Gag and Pol; a vector encoding a Rev packaging element; and a vector encoding an envelope protein are combined. CaCl2 is added to plasmid mixture. 2X HBS can be added to the above transfection mix with stirring. The transfection mix is incubated and added to the cells in a petri dish. The cells can be incubated in a CO2 incubator at 37° C. with a 5% CO2 atmosphere. Post-transfection, the calcium phosphate-containing medium is replaced with complete media (DMEM+10% FBS) and incubated in CO2 incubator at 37° C. with a 5% CO2 atmosphere. Spent media from confluent transfected cells (e.g., LentiX 293T) are filtered. Aliquots of the filtered spent media with the lentivirus can be frozen and stored.

Viral vectors for specific clones can be produced in cells (e.g., LentiX 293T). For example, the cells are seeded in a well of a well plate. After 24 hours, cells are co-transfected with the plasmid clone; one or more packaging elements or envelope proteins; and transfecting with a transfection reagent (e.g., Lipofectamine LTX (Invitrogen)). After incubation, media is replaced, and cells are cultured in complete media. Cell supernatants are collected, filtered, frozen, and stored.

Viruses can be titered by seeding cells in a well of a well plate and culturing in complete media (e.g., DMEM+10% FBS). Serial dilutions of virus can be added after removing majority of the spent media from the wells and incubated. Complete media can be added and incubated. Spent media is removed, replaced with complete media containing a selection compound (e.g., puromycin), and incubated. The cells are inspected for viability under the microscope and colonies are counted to calculate the infectious unit/ml.

### GFP Reporter Cell Lines

Cells can be seeded in the well of a well plate and grown in complete media (e.g., DMEM). For example, a GFP reporter plasmid carrying LTR-GFP and a resistance marker (e.g., blasticidin S-resistance (BSR) gene) is transfected in the cells (e.g., LentiX 293T) and incubated. Transfected cells are selected for marker resistance (e.g., blasticidin S), exchanging media (e.g., DMEM) with the marker every 3 days. Cells are trypsinized and cells are serially diluted in well plates. After incubation, single colonies are screened after expansion.

For confirming viral integration, gDNA is isolated. Tat amplicons are subcloned and sequenced. Tat transcriptional activity is measured in a subculture of each clonal cell line. Cells culture in well plates are transfected with wild-type Tat expression vector and cultured. Transactivation-induced GFP expression is evaluated by epifluorescence microscopy. The clonal reporter cell lines are propagated, frozen, and stored.

### Cell Lines and Libraries

Cells (e.g., LentiX 293T/LTR-GFP) can be transduced with the Her2 variant viral library at a multiplicity of infection (MOI) of 0.1. After infection, cells are cultured and maintained in complete media supplemented with a selection agent (e.g., puromycin). Confluent cells are harvested, counted, and washed once with 1X PBS before fixing and isolating gDNA for NGS of the Her2 amplicon.

Cells (e.g., Jurkat/LTR-GFP) are seeded and transduced with 0.1 MOI of the viral library. After transduction, the cells are selected for viral survival in media (e.g., RPMI 1640+10% FBS), supplemented with a selection agent (e.g., puromycin). Next, the cells are counted, washed with 1X PBS, and fixed for flow sorting and subsequent isolation of gDNA.

For performance evaluation of the high-throughput cellular molecular function assay system and method, random variants of Her2, as well as empty vector and wtHer2 are stably expressed in cells (e.g., LentiX 293T/LTR-GFP). Cells are seeded in a well of a well plate and incubated. Cells are transduced with a virus and selected and maintained in complete media with the selection agent (e.g., puromycin). Cells are harvested and analyzed by flow cytometry to assess for LTR transactivated GFP expression. The same stable cell lines can be created in Jurkat/LTR-GFP cells. Selected clones for empty vector, wtHer2, and Her2 variants are frozen and stored.

In some embodiments, one fourth of the LentiX293T/LTR-GFP and one tenth of the Jurkat/LTR-GFP cells are harvested, gDNA is isolated and sequenced to evaluate library representation before Flow Sorting. The remaining cells are fixed in 2% paraformaldehyde/PBS, washed twice with 1X PBS and resuspended in 1X PBS for analysis by flow sorting (e.g., Sony 800S Cell sorter). Cells can be sorted into three bins of GFP signal intensity (low-GFP, mid-GFP and high-GFP) gated with threshold determined for cells stably expressing wt-Tat for maximal transactivation of LTR-GFP, and cells stable expressing a Her2 variant or empty vector for low background of basal transactivation of LTR-GFP.

For deep sequencing, primers can be designed to flank the Her2 targeted region from gDNA and incorporate the NGS sequencing adaptors. gDNA is amplified by PCR. NGS libraries for each sample category can use 10 NGS library forward primers and 1 NGS library reverse primer. The forward primers can be common for all the sample categories and the reverse primer being unique for each sample. The Her2 amplicons are pooled and purified (e.g., gel extraction). All the samples are pooled and sequenced (e.g., Novaseq 6000 sequencing platform). Samples are sequenced (synthetic dsDNA Her2 variant library, plasmid library, selected cell libraries in cells (in duplicate), and flow sorted low-GFP, mid-GFP, and high GFP cells for each cell line (in duplicate).

### Bioinformatics

Provided herein are databases that comprise ERBB2 variants identified by the method described herein that are implicated in the progression of cancer. The sequencing data from the methods described herein is processed and stored in a database. For example, paired-end reads can be processed with a multistep bioinformatic pipeline (e.g., BaseSpace) and resulting reads in files (e.g., bcl) are converted into processed files (e.g., FASTQ). Read quality can be assessed by an algorithm (e.g., FASTQC). Paired end reads for all samples are merged together (e.g., FLASH) to build complete Her2 contigs. Contigs are quality trimmed (e.g., Trimmomatic). Adapters are trimmed, and barcodes are isolated (e.g., CutAdapt) and barcodes are grouped (e.g., Starcode). The sequence reads are demultiplexed into subsets of read sequences for each cell clone based on unique barcodes with a custom script (e.g., Python) that processes the output of grouped barcodes. Resulting reads are then aligned to the Her2 cDNA. The file with nucleotide variants are called for each subset of Her2 contigs (cell clones) and output as a file. Custom Python scripts can be used to identify the amino acid substation for the output files, the number for reads for each barcode in each sample, and the barcodes groups for cells with the same amino acid substitutions. This library can be used in scripts that gathered the information for each variant from the output files. Read counts and read depths for each barcode and each amino acid substitution in each sample are normalized to the number of reads/million and activity is measured by the percentage of GFP+ reads for each barcode and each variant.

Statistics can be calculated for each variation. In some instances, there are n cell lines (biological replicates) and each cell line has m technical replicates. For each barcode (group) in a sample, the percentage of the number of reads in the GFP+ group vs the total number of reads in both GFP+ and GFP− groups can be calculated, denoted as h ratio (h∈[0,1]). In some instances, a high h percentage for wild type, while a low h percentage suggests a variant. Then for each variant, calculate the averaged h ratio for all the barcodes assigned to the same variant, denoted as a variant level summary score. In some instances, use a one sample t-test to evaluate 1) whether the variant has a significantly different number of reads in the GFP+ group compared with the GFP− group within a technical replicate, and 2) whether the variant has a significantly different number of reads in the GFP+ group compared with the GFP-group among different cell lines based on biological replicates (null hypothesis H0: h=0.5).

In some instances, classify variants with high h percentage as wild type and a low h percentage as a LOF variant. To estimate type I error for the classification, in some instances compile a list of true variants with wild type transcriptional activity and true LOF variants with low activity. Then fit their h percentages with a beta distribution as the null distribution. Specifically, for the wild type detection, in some instances, use the true variant as the null, and vice versus, for the variant detection, use the wild type as the null. Moment estimators are used for estimating the model parameters. The p values for different cell lines are combined using Fisher's method into a global test p value.

Performance metrics of accuracy, sensitivity, specificity, positive predictive value and negative value can be based upon standard formulas.

Figures can be prepared with PowerPoint, Excel, FlowJo, and Pymol. Bin, Bar, and Pie plots, as well as saturating mutagenesis heatmaps generated with Excel. Values for saturating mutagenesis heatmaps and 3D surfaces plots can be generated with custom python scripts. 3D surface plots for the amino acid tolerance at each position represented accuracy of physiochemical properties as color gradients and indicate the highest accuracy. Accuracy is a standard formula and is calculated for groups of amino acids with similar physiochemical properties. Solvent accessible surface area (SASA) can be calculated for the Her2 structure. Residues are considered buried if less than 10% of surface area is exposed to solvent.

For example, the MCC formula is calculated with the following data definitions for large hydrophobic amino acids, at a position in Her2 as an example: If either Phe, Tyr, or Trp have >50% activity they are true positives and if the other amino acids have <50% activity they are true negatives. If either Phe, Tyr, or Trp have <50% activity they are false positives and if the other amino acids have >50% activity they are false negatives. Also consider the wild type amino acid to be a true positive when it is in the physiochemical group, and as a true negative when it is not. The MCC captures the tolerance for types of amino acids at each position and when mapped the surface of the 3D structure, is a new visual mining approach to reveal the spatial relationships of amino acids tolerances and their relevance to other Her2 functions.

### Example 1: ERBB2 Plasmid Library

ERBB2 (HER2) is an oncogene implicated in the progression of cancers. Further, activating missense variations in the tyrosine kinase domain and juxtamembrane region of ERBB2 (HER2) may be a driver of variations in multiple tumor types. Historically there has been a lack of uniformity in the assays used by disparate groups for characterizing these variants, resulting, at least in part, in a lack of clarity regarding the impact of each variant on protein function, oncogenic potential, or both.

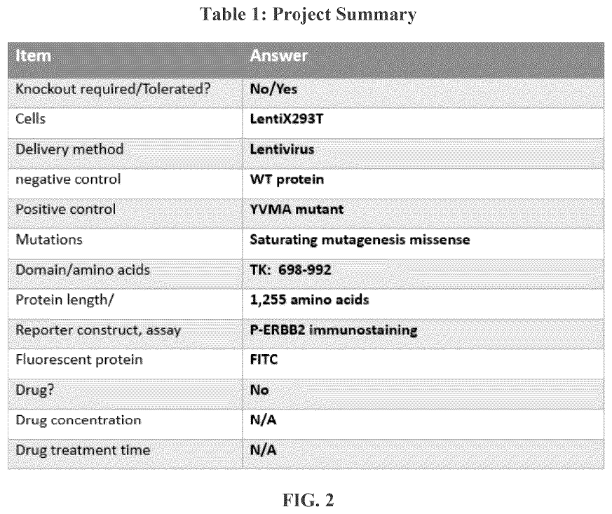

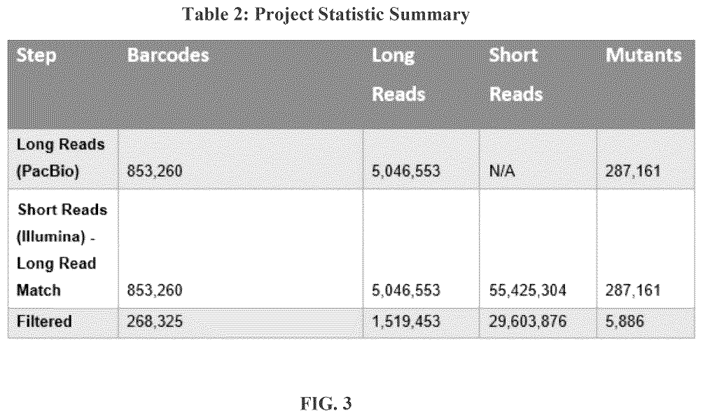

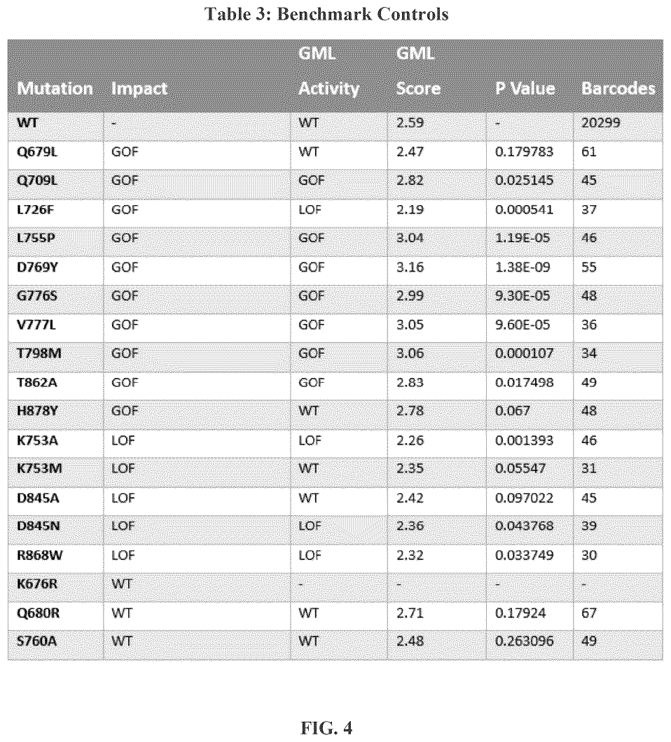

The parameters for the project are summarized in the Table in FIG. 2. The ERBB2 plasmid library that was produced contained 99% of the expected 5,966 variants, with most missed variants coming from 4 positions. The ERBB2 library contained the tyrosine kinase domain (residues 715-992), as well as the JM region (residues 679-714) that was not part of the original design. The p-Her2 GML results were filtered, removing variants with low read numbers (<1 barcode), and those not designed in the original experiment. The filtered set had measurements for 5,886 (99%) of the variants designed in the experiment (Table in FIG. 3).

### Example 2: Results And Interpretation

The plasmid library was sequenced with PacBio CCS long reads (n=1,519,453) and variants were called in the Heligenics pipeline (Table in FIG. 3). Next, a lentiviral library was created and transduced into dox-inducible LentiX293T cells. Expression of variant protein was induced with doxycycline for 72 h prior to cell harvest. Harvested cells were fixed, permeabilized, and then immunostained with antibodies directed towards Her2 and p-Her2 (Tyr1248). The immunostained cell library was then flow sorted, firstly for Her2 expression and then into 4 bins of graded fluorescence based on p-Her2 expression. After sorting, the GML was sequenced by next generation sequencing (NGS) and filtered, which produced 29,603,876 short reads for calculating unique molecular identifier (UMI)-barcode frequencies in each bin. Of the 15,845,059 sorted barcoded cells, there were 268,325 unique barcodes for an average of 46 barcodes/variant. Relative activity levels were calculated and variant activities were classified as wild type (WT), reduced function (RF) or gain-of-function (GOF) by comparing each variant to WT with a two-sample t-test. The method described herein was optimized to primarily identify GOF variants, and not RF variants.

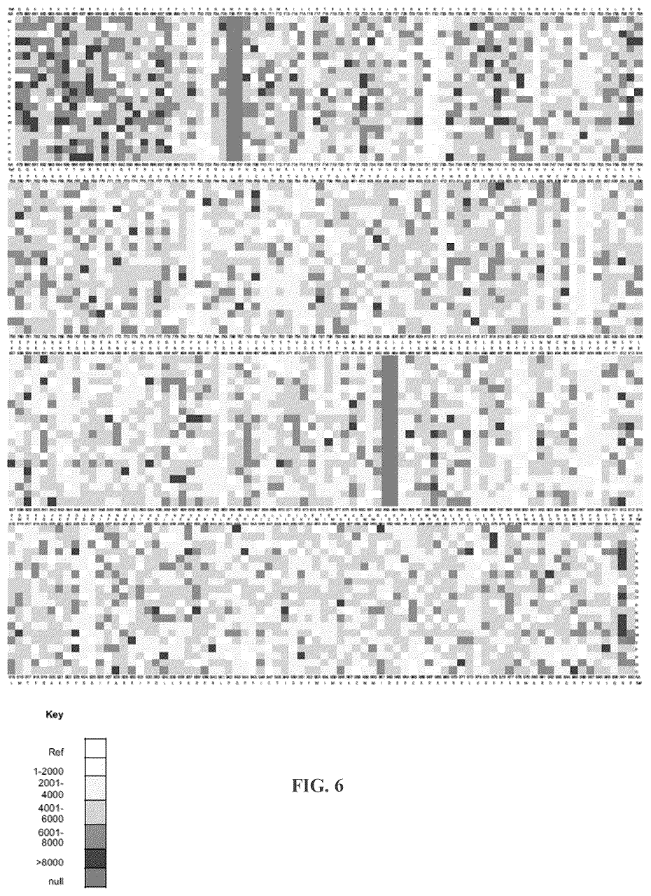

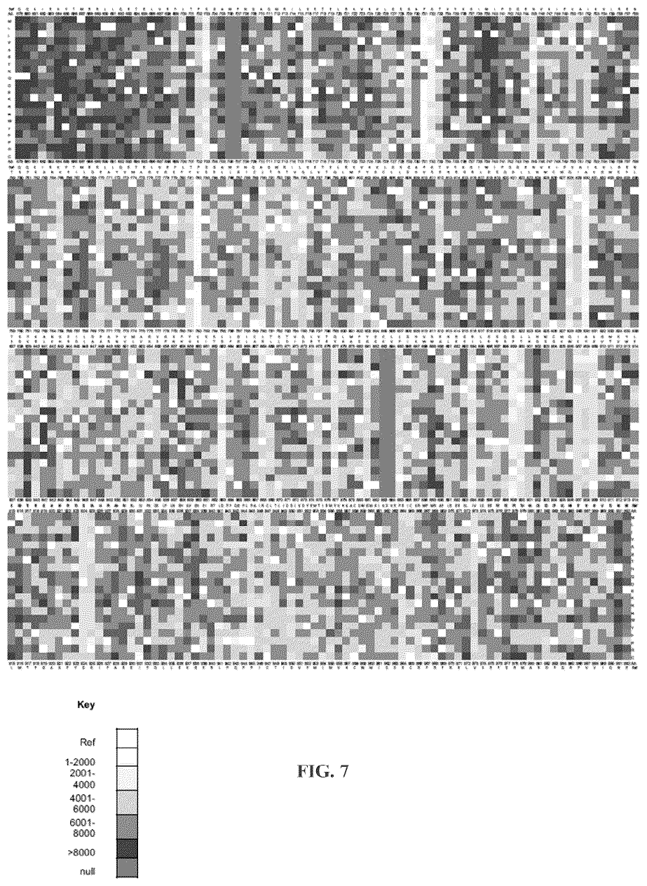

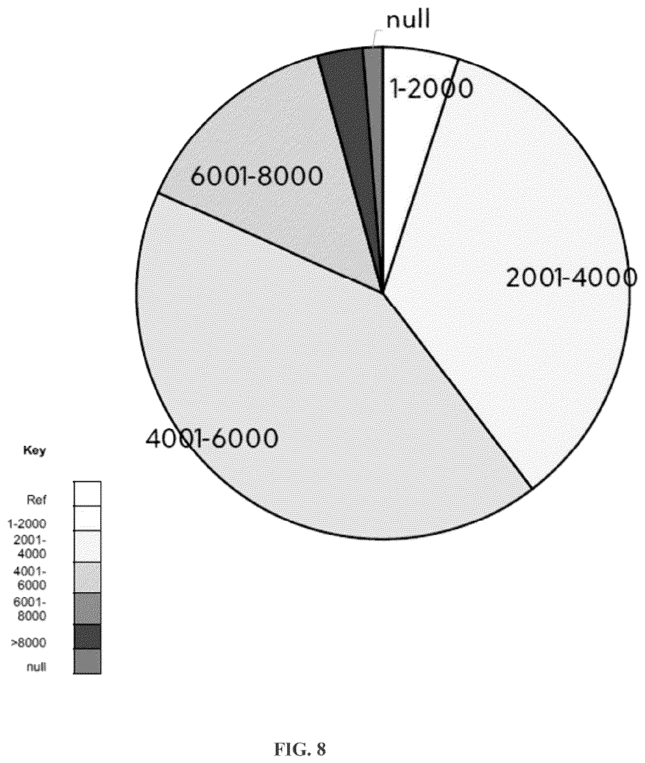

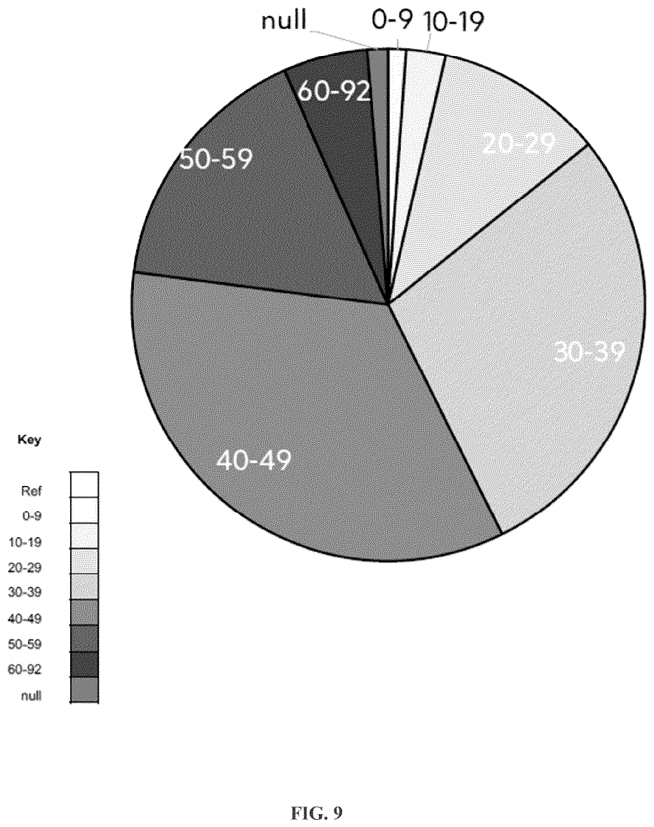

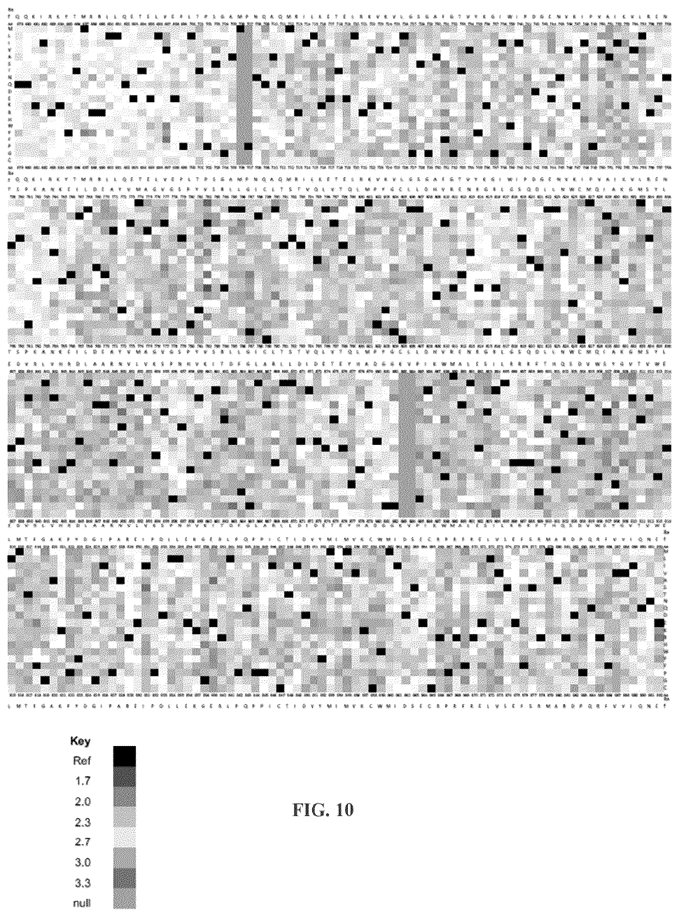

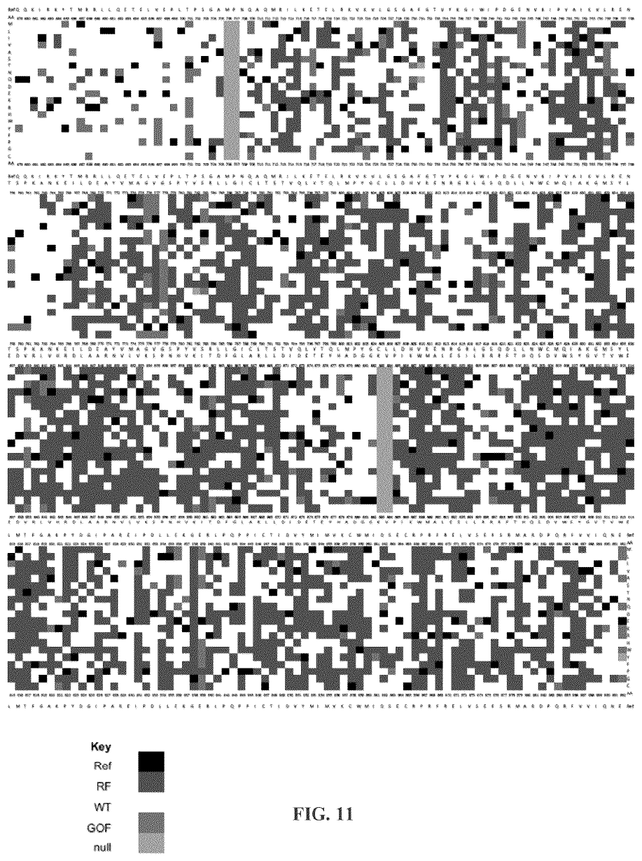

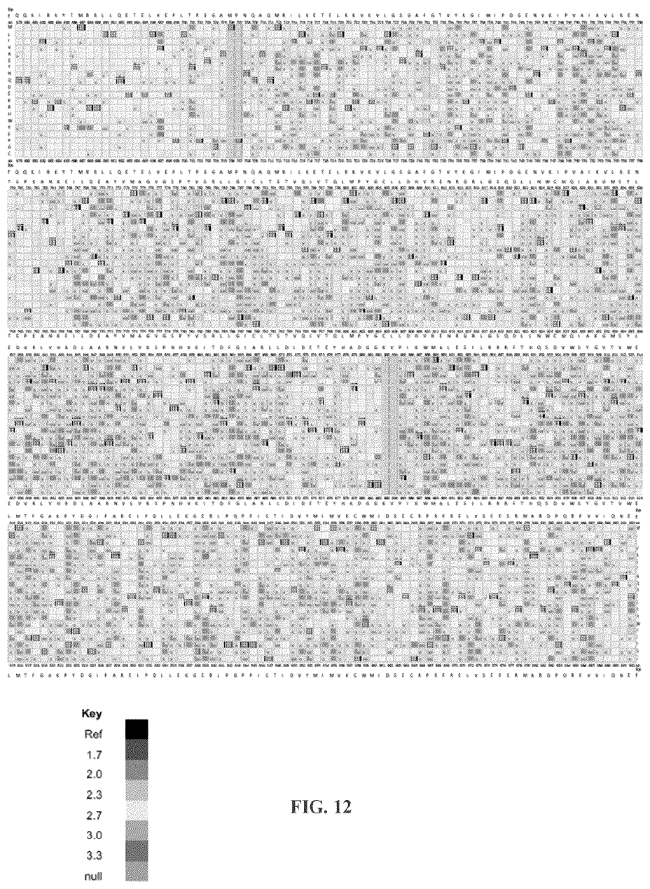

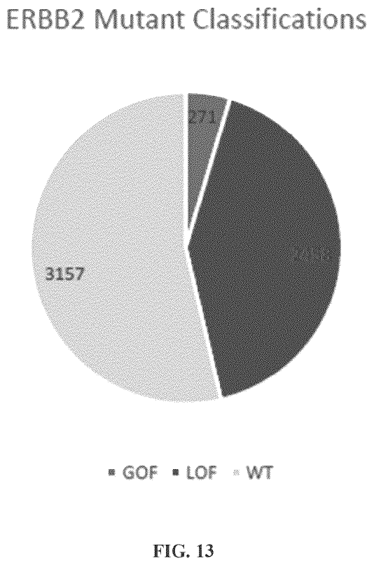

The number of reads and barcodes associated with each variant are shown in heatmaps (FIGS. 6-7) and read and barcode counts for all variants are shown in pie charts (FIGS. 8-9). A saturating mutagenesis heatmap of p-Her2 activity, a statistical test for each variant, and an overlay combining both data are shown in FIGS. 10-11. These data show which variants are WT, GOF, and RF. The number of variants that passed statistical significance for either GOF or RF activity was quantified (FIG. 12). Approximately 4.6% of variants were GOF, 41.8% were RF, and 53.6% were WT using p<0.05 as a classification threshold (FIG. 13). A list of the variants, number of barcodes, their relative activities, classification, and p values are reported in the Table in FIG. 4.

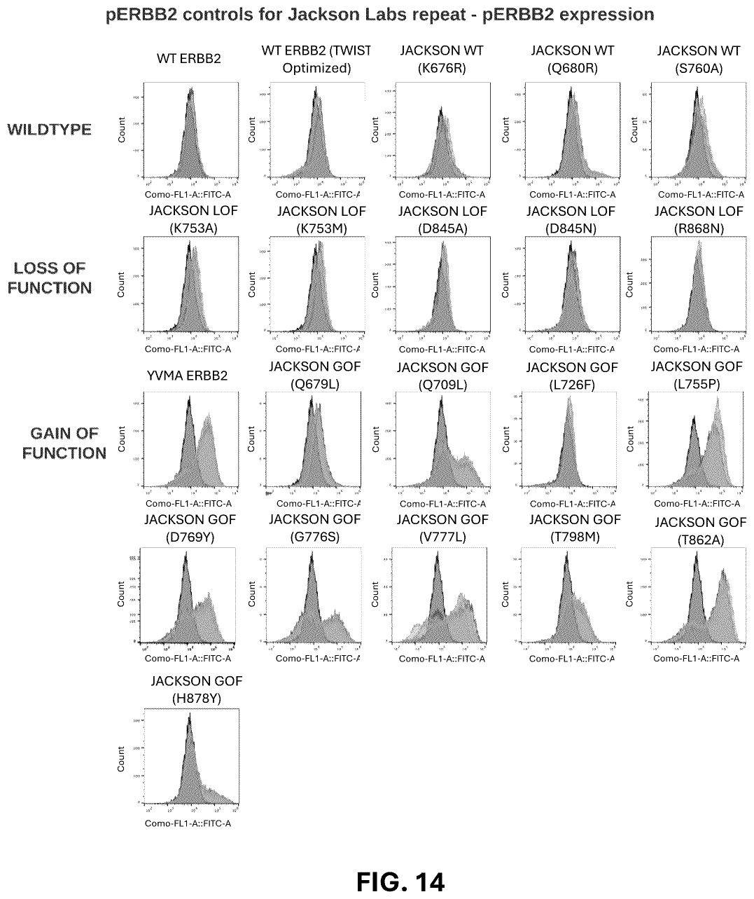

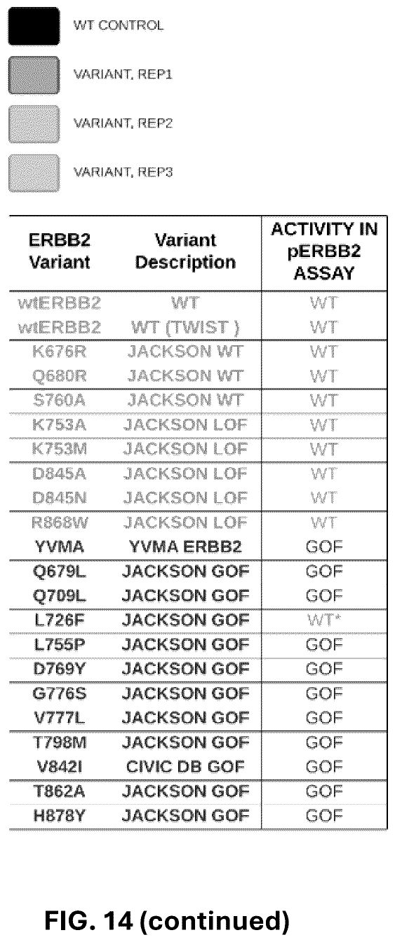

17 benchmark control variants were tested; 1 additional variant provided (K676R) was outside of the tested region. Stable cell lines expressing each of these variants were generated and their activity was measured individually by flow cytometry (FIG. 14). 3 variants (Q679L, H878Y, L726F) were identified that had some additional considerations. L726F was not a GOF variant determined in flow cytometry profile and both H878Y and Q679L had GOF activity in the flow profile, but this was modest when compared to most other GOF variants (FIG. 14). The results for these variants were generally in alignment with other methods. HPAFII cells expressing Q679L exhibit no increase in p-Her2 while its expression in HPNE cells promotes elevated levels of p-Her2, suggesting cell line specificity for autophosphorylation of this variant. Expression of H878Y in HEK293, BaF3, and BEAS-2B cells promoted only a small increase in p-Her2 expression relative to cells expressing WT protein. Finally, MCF10A cells expressing L726F exhibit reduced phosphorylation at multiple sites relative to MCF10A cells expressing WT protein.

To compare these results with other high throughput approaches, multiplexed assay of variant effect (MAYE) enrichment screen for oncogenic variants were performed. In some implementations, only oncogenic variants G776S and D769Y were significantly enriched in the MAYE assay, which in this instance, had an accuracy of 41%, despite the high reproducibility among replicates with a R2 of 0.94 for all variants tested.

Her2 phosphorylation was measured at a single site and other pathways may contribute to GOF or RF activity, which could be tested in separate assays.

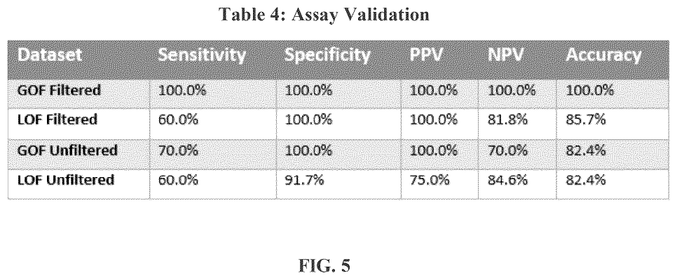

Given that the p-Her2 assay was designed to identify GOF variants with elevated p-Her2, exceptional assay performance was observed when the 3 benchmark variants lacking elevated Her2 phosphorylation were removed, similar to published results for HIV Tat [5]. When the GOF Her2 activity data were compared benchmark data, it demonstrated high accuracy (Acc=100%), with other performance metrics=100% (Table in FIG. 5). When the RF Her2 activity data were compared to the benchmark data it demonstrated high accuracy (Acc=88%), with other performance metrics in Table 4.

Modestly reduced performance was observed when the 3 benchmark variants were included in the analysis. When the GOF Her2 activity data were compared to benchmark data it demonstrated high accuracy (Acc=82%), with other performance metrics in Table 4. When the RF Her2 activity data were compared to the benchmark data it also demonstrated an accuracy=82%, with other performance metrics in Table in FIG. 5.

Two RF variants (D845A, K753M) were correctly identified in the assay as RF but did not pass statistical significance for the RF classification. However, it should be noted that this particular assay was not designed to detect LOF variants and these variants were just below the statistical significance threshold. These variants had p values of 0.1-0.05 and were the only LOF variants that did not validate.

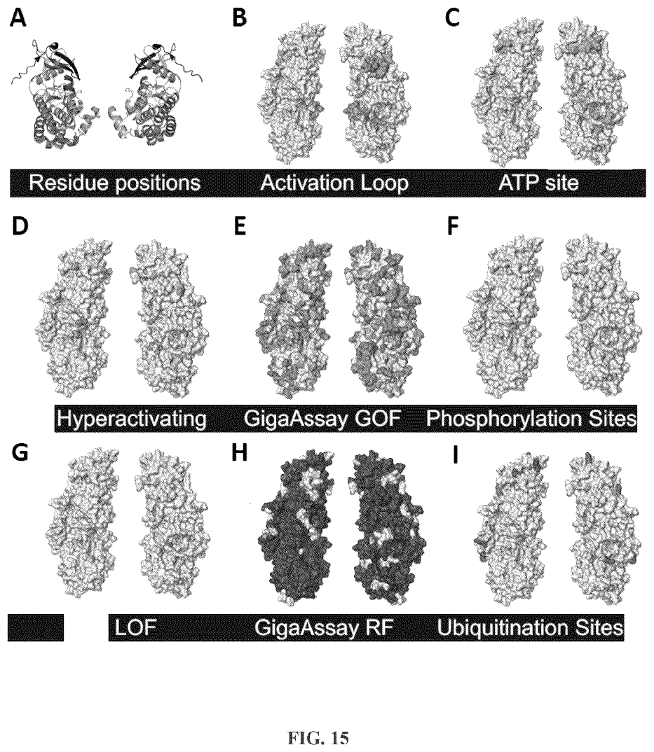

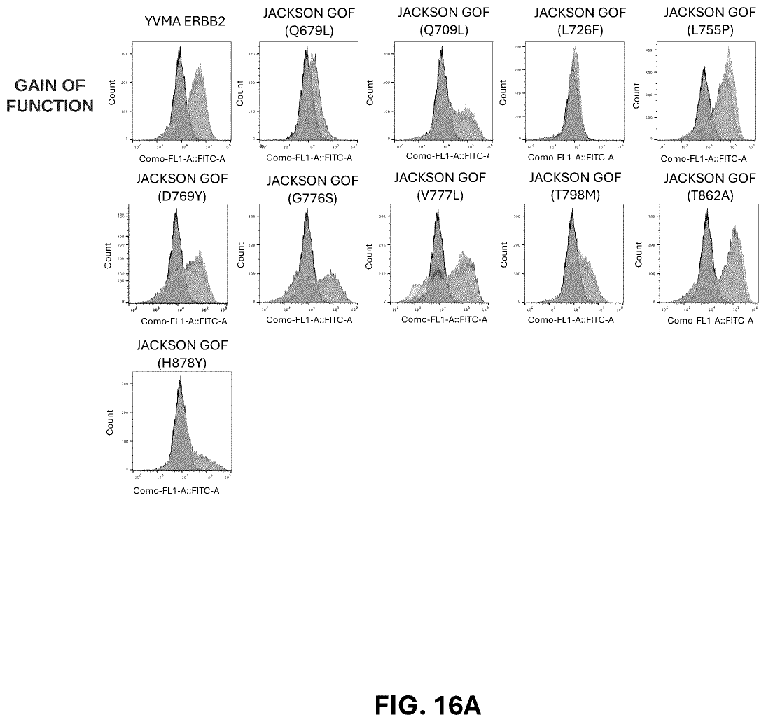

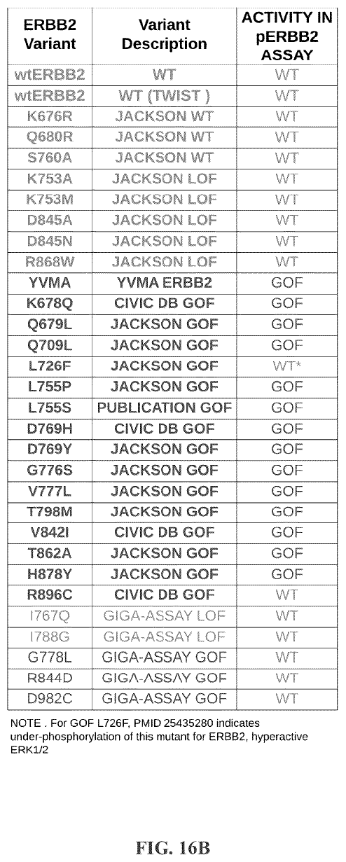

The variants were plotted onto the surface of a crystal structure of the tyrosine kinase domain of Her2 (PDBid: 3PPO). A ribbon diagram shows a rainbow from the N- to C-terminus (blue to red; FIG. 15A). Regions of the kinase activation loop and ATP binding site are shown for comparison (FIGS. 15B-15C). The 271 GOF variants are shown in FIGS. 15D-15E. Phosphorylation sites from PhosphoSite are shown in FIG. 15F. The LOF variants and RF variants determined with the method described herein are shown in FIGS. 15G-15H. Ubiquitination sites from PhosphoSite are in FIG. 15I. Each position has 19 measured amino acid substitutions. For GOF at least one substitution with a GOF activity (p<0.05) and RF at least one substitution with a RF activity (p<0.05). It is common to see positions that have substitutions with both RF and GOF activities (see heatmap in FIG. 6). For these plots, counting occurs only if there is a variation at the position and not which of the 19-amino acid are substituted. Only statistically significant variants from the method described herein are plotted (p<0.05). There was no structure for human Her2 that have both the tyrosine kinase domain and JM region. Therefore, for structural evaluation of variants in the JM region a predicted structure from Alphafold V2.0 was used. The GOF and RF variants in the JM region are shown on the Her2 structure predicted with Alphafold V2.0 (FIG. 15).

While exemplary embodiments have been shown and described herein, such embodiments are provided by way of example only. Numerous variations, changes, and substitutions are within the scope of the present disclosure. It should be understood that various alternatives to the embodiments described herein may be employed. It is intended that the following claims define the scope of the disclosure and that methods and structures within the scope of these claims and their equivalents be covered thereby.

1. A method of identifying an Erb-B2 Receptor Tyrosine Kinase 2 (ERBB2) polypeptide variant implicated in a cancer, the method comprising:
(a) providing a plasmid library for expression of a library of ERBB2 polypeptide variants, wherein each plasmid in the plasmid library comprises:
(i) a promoter;
(ii) a polynucleotide sequence encoding an ERBB2 polypeptide variant, from the library of ERBB2 polypeptide variants, that is operably coupled to the promoter, wherein each ERBB2 polypeptide variant in the library of ERBB2 polypeptide variants independently and substantially comprises a single amino acid substitution in a region of an ERBB2 polypeptide ranging from residue 679 to residue 992 of SEQ ID NO: 1; wherein the library of ERBB2 polypeptide variants comprises ERBB2 polypeptide variants that collectively have an amino acid substitution of substantially all 20 amino acids at substantially every amino acid residue in the region of the ERBB2 polypeptide ranging from residue 679 to residue 992 of SEQ ID NO: 1; and
iii) a barcode, wherein each plasmid in the plasmid library independently has a different barcode associated with the polynucleotide sequence, in the plasmid, encoding an ERBB2 polypeptide variant;
(b) contacting a plurality of mammalian cells with the plasmid library, wherein the contacting results in expression of a single ERBB2 polypeptide variant among the library of ERBB2 polypeptides on a surface of a single mammalian cell among the plurality of mammalian cells, thereby making a plurality of mammalian cells expressing the library of ERBB2 polypeptide variants, wherein a subset of the plurality of mammalian cells expressing the library of ERBB2 polypeptide variants has an ERBB2 polypeptide variant that is phosphorylated to a greater extent than a wildtype ERBB2 polypeptide expressed on the surface of a mammalian cell, and wherein the ERBB2 polypeptide variant that is phosphorylated to a greater extent than the wildtype ERBB2 polypeptide is the ERBB2 variant that is implicated in the cancer;
(c) identifying the subset of mammalian cells expressing the ERBB2 polypeptide variant that is phosphorylated to a greater extent than the wildtype ERBB2 polypeptide; and
(d) sequencing the barcode of the plasmid present in each mammalian cell of the subset of mammalian cells, thereby identifying the ERBB2 polypeptide variant implicated in the cancer.

2. The method of claim 1, wherein the cancer comprises a carcinoma, an ovarian cancer, a stomach cancer, a bladder cancer, a salivary cancer, or a lung cancer.

3. (canceled)

4. The method of claim 1, further comprising detecting the presence of the ERBB2 polypeptide variant identified in (d) from a sample obtained from a subject.

5. The method of claim 4, further comprising diagnosing the subject as having the cancer, or being at risk of developing the cancer.

6. The method of claim 4, further comprising administering an anticancer treatment to the subject, optionally based on the presence of the ERBB2 polypeptide variant identified in (d) from the sample obtained from the subject.

7. The method of claim 6, wherein the anticancer treatment is effective against a cancer cell expressing the ERBB2 polypeptide variant.

8. The method of claim 1, wherein the identifying in (c) comprises contacting the plurality of mammalian cells expressing the library of ERBB2 polypeptide variants with an agent that binds to a phosphorylated ERBB2 polypeptide.

9. (canceled)

10. The method of claim 8, wherein the identifying of (c) further comprises performing an immunoassay using the agent that binds to phosphorylated ERBB2, wherein the agent that binds to phosphorylated ERBB2 is an antibody directed against the phosphorylated ERBB2 polypeptide

11. The method of claim 10, wherein the identifying of (c) further comprises performing cell sorting based on the immunoassay.

12. The method of claim 11, wherein the cell sorting is fluorescence activated cell sorting.

13. The method of claim 1, wherein the promoter is an inducible promoter.

14. The method of claim 13, wherein the inducible promoter is a doxycycline-inducible promoter.

15. The method of claim 1, wherein a plasmid in the library of plasmids is a viral plasmid.

16. The method of claim 15, wherein the viral plasmid is a lentiviral plasmid or an adeno-associated viral plasmid.

17. The method of claim 15, further comprising packaging the viral plasmid in a viral capsid prior to the contacting of (b), thereby producing a virion that contains the viral plasmid.

18. The method of claim 17, wherein the contacting of (b) comprises contacting a mammalian cell of the plurality of mammalian cells with the virion.

19. The method of claim 1, wherein the plurality of mammalian cells comprise HEK 293 cells.

20. (canceled)

21. The method of claim 1, further comprising stably-expressing the ERBB2 polypeptide variant implicated in cancer identified in (d) in a mammalian cell and measuring an activity of the ERBB2 polypeptide when stably expressed, wherein at least 80% of the variants identified in (d) display higher activity when stably expressed, as compared to stably expressed wildtype ERBB2.

22.-24. (canceled)

25. A database that comprises the ERBB2 polypeptide variant identified by the method of claim 1.

26. A composition that comprises the ERBB2 polypeptide variant identified by the method of claim 1.


SEQUENCE LISTING (3 sequences)

SEQ ID NO: 1 - protein, 1255 aa, Homo sapiens [sequence omitted]

SEQ ID NO: 2 - protein, 4 aa, Homo sapiens
YVMA

SEQ ID NO: 3 - protein, 314 aa, Homo sapiens
QQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNE